(19) United States
(12) Patent Application Publication
(10) Pub. No.: US 2019/0192058 A1
(43) Pub. Date: Jun. 27, 2019

(54) METHOD AND APPARATUS FOR SAMPLING BLOOD GLUCOSE LEVELS

(71) Applicant: Dottli Oy, Espoo (FI)

(72) Inventors: Mikko TASANEN, Tampere (FI); Vesa KEMPPAINEN, Espoo (FI)

(21) Appl. No.: 16/327,885

(22) PCT Filed: Aug. 10, 2017
(86) PCT No.: PCT/EP2017/070314

§ 371 Date: Feb. 25, 2019

(30) Foreign Application Priority Data
Aug. 26, 2016 (FI) 20165638

(51) Int. Cl. A61B 5/145; A61B 5/00

(52) U.S. Cl. CPC A61B 5/14532; G16H 20/17; A61B 5/7275

(57) ABSTRACT

A method for sampling momentary blood glucose levels, especially in the context of self-performed measurements conducted by a diabetes patient or by a user who assists the patient. Also provided are methods for estimating long-term blood glucose levels and glycated hemoglobin (HbA1c) concentration and methods for estimating blood glucose variance from the data obtained with the sampling method. The sampling method is based on a measurement of momentary blood glucose level data at randomly distributed sampling moments. The advantage obtained with this method is that averages calculated from measurements conducted at randomly distributed sampling moments are independent of periodically recurring events in the user's daily or weekly routines. The systematic errors arising from such routines can therefore be avoided.

### CROSS-REFERENCE TO RELATED APPLICATIONS

The present application is a National Stage Application of PCT International Application No. PCT/EP2017/070314 (filed on Aug. 10, 2017), under 35 U.S.C. § 371, which claims priority to Finnish Patent Application No. 20165638 (filed on Aug. 26, 2016), which are each hereby incorporated by reference in their respective entireties.

### TECHNICAL FIELD

The present disclosure relates to methods for monitoring health risks associated with diabetes. More particularly, it relates to a method for sampling blood glucose levels and using these sampling results to estimate the long-term blood glucose level, and/or to estimate the glycated hemoglobin (HbA1c) concentration, and/or to estimate blood glucose variance.

### BACKGROUND

Elevated blood glucose levels significantly increase the risk of additional diseases associated with diabetes, such as peripheral vascular disease, diabetic nephropathy and diabetic retinopathy. Monitoring of blood glucose levels is therefore an important aspect of diabetes treatment.

In the long term, the primary variable of interest is usually the average blood glucose level. In this disclosure the term “long-term blood glucose level” refers to the average value of the blood glucose level during a time period on the order of one month or more. The long-term blood glucose level can be expressed as a concentration.

Another variable of interest is the amount of variation in the blood glucose level, either during the course of a short period, such as one day, or during longer periods. In this disclosure the term “blood glucose variance” refers to the variance in blood glucose levels during a given period.

A detailed presentation of short-term blood glucose variance may involve a time-dependent profile, such as an ambulatory glucose profile. To calculate this profile, measurements may be conducted over several days and every measurement is categorized according to the time of day when it was obtained. For example, if an hourly resolution is chosen, every measurement performed between 10 o'clock in the morning and 11 o'clock in the morning would be categorized as a 10-11 measurement. In the ambulatory glucose profile, the median and variance of blood glucose levels sampled (repeatedly over the course of several days) in each hourly profile interval is presented as a function of the time of day. The time resolution used in the calculation of the profile can be shorter or longer than an hour, for instance 30 minutes or 2 hours.

Long- and short-term blood glucose monitoring can rely on multiple data sources. Many diabetes patients monitor their blood glucose levels through daily blood glucose level measurements which they perform themselves. Periodic check-ups at hospitals or the like provide more detailed data. Both of these data sources rely on sampling, that is, measurements performed at regular or irregular intervals.

However, aggregating sampled blood glucose levels into reliable estimates of long-term blood glucose levels or blood glucose variance is not a simple exercise. There are practical limits to how often a diabetes patient can conduct blood glucose level measurements, and these limits may easily produce systematic errors.

Most patients perform a blood glucose level measurement 1-12 times per 24 hours, which equals 0.04-0.5 measurements per hour. This is a very low sampling frequency for a volatile variable such as blood glucose level, whose value can change by 200% per hour. Furthermore, even if patients may be instructed to distribute measurements evenly throughout the day, the timing of self-performed measurements tends to be determined by recurring daily routines and habits. The same sampling moments are easily repeated from one day to the next and their timing in relation to meals often remains constant. Meals significantly influences blood glucose levels. The sampling schedule may also be influenced by how the patient is feeling at certain hours of the day, since many patients perform a measurement to check whether or not a certain feeling is caused by an unusually low or high blood glucose level.

For all of these reasons, direct averaging of self-performed measurements does not provide reliable estimates of either long-term blood glucose levels or blood glucose variance because several sources of systematic error may be present. These problems relating to sampling can to some extent be circumvented with continuous glucose monitoring. However, continuous monitoring requires implantation of sensors under the patient's skin, which is quite costly and can be troublesome to the patient. Furthermore, even sensors which perform continuous glucose monitoring have to be periodically calibrated against and externally measured, regularly sampled blood glucose levels.

As far as long-term blood glucose level measurements are concerned, an alternative to periodic sampling is to measure hemoglobin concentrations. Some of the hemoglobin in red blood cells reacts with the glucose present in the blood stream, forming glycated hemoglobin (HbA1c). The fraction of hemoglobin which undergoes this reaction is directly proportional to glucose concentration. Glycated hemoglobin is not present in newly formed blood cells, and it is not formed by any other process than reaction with glucose. Consequently, since the average lifespan of red blood cells is approximately 3 months, a momentary measurement of HbA1c hemoglobin concentration (expressed, for example, as a percentage of all hemoglobin or with the unit mmol/mol) is a reliable indicator of the long-term blood glucose level during the 1-3 months preceding the measurement.

However, HbA1c concentration can only be measured with relatively complex laboratory equipment. A typical diabetes patient may therefore have his or her HbA1c value checked just 2-4 times a year in conjunction with periodic check-ups at the hospital or the like. Various methods have been proposed for calculating an estimate for the HbA1c value from momentary blood glucose measurements which the patient can perform on her own. Such estimates allow the patient to follow her HbA1c value continuously. The primary reason for estimating the HbA1c value (an indicator of long-term blood glucose level, but not a true measure of said level) is that this value is widely known and used indicator. As such, it is more familiar to patients and healthcare professionals than the long-term blood glucose level itself.

For the reasons given above, there is a need for reliable methods for sampling momentary blood glucose levels without systematic error.

U.S. Patent Publication No. US2010330598 A1 discloses a method for estimating both long-term blood glucose levels and HbA1c values from momentary blood glucose measurements. Blood glucose values are sampled according to a predetermined sampling schema and each value is weighted with a coefficient based on the context of that specific measurement. The long-term blood glucose level and HbA1c are then estimated from these weighted measurements. A problem with this method is that its reliability depends on the stability of the patient's diabetes and the accuracy of the weightings. The method would not give reliable estimates for T1 diabetics or T2 diabetics whose glucose levels fluctuate significantly. The reliability of general weighting coefficients varies from patient to patient because each patient has idiosyncratic daily habits.

European Patent Publication No. EP2939159 A2 discloses a method for estimating blood glucose variances. The document discusses both continuous glucose monitoring and sampling. The sampling method is based on analysing the patient's sampling routines, identifying gaps where glucose level measurements are typically lacking during the day, and prompting the patient to perform measurements at those times. A problem with this method is that, since the number of sampling moments in each day is limited, gaps will typically be filled by a measurement in the middle of the gap and the sampling will still be determined by the patient's daily routines. Even with gap-filling prompts the patient's daily sampling schedule is likely to be repetitive. The glucose level will repeatedly go unobserved on some hours of the day, which increases the risk of systematic errors.

### SUMMARY

An object of the present disclosure is to provide a method and an apparatus for implementing the method so as to alleviate the above disadvantages. The objects of the disclosure are achieved by a method and an apparatus which are characterized by what is stated in the independent claims. The preferred embodiments of the disclosure are disclosed in the dependent claims.

The disclosure is based on the idea of measuring momentary blood glucose level data at randomly distributed sampling moments. The randomization is performed by a computer. The advantage over sampling methods where the sampling moments are determined by a patient, or another user who assists the patient, is that averages calculated from measurements conducted at randomly distributed sampling moments are independent of periodically recurring events in the user's daily or weekly routines. In other words, the systematic error sources described above can be avoided, or their influence at least mitigated, by prompting users to perform a measurement of momentary blood glucose level at sampling moments which have been distributed randomly across a measurement period by a computer.

With regard to estimates of long-term blood glucose levels and HbA1c concentration, randomization is an easier and more reliable way to negate the influence of users' periodic routines than the assignment of arbitrary weighting coefficients based on the time of day. Randomization requires no guesswork concerning how well a particular patient's routines and meal-dependent variation in blood glucose levels might correspond to those represented in the coefficients, or how stable it might remain over time.

With regard to blood glucose variance, randomization is a more reliable way to obtain representative blood glucose level measurements than a method based on filling the gaps. Again, the advantage of randomization is that it is completely independent of the user's preferred sampling schedule or daily routines. Randomly distributed sampling moments therefore produce measurement data which is better suited for estimating variance than data obtained by filling gaps in the user's normal sampling schedule.

### DRAWINGS

In the following the disclosure will be described in greater detail with reference to the accompanying FIGS. 1 and 2.

FIG. 1 presents a flowchart of the methods presented in this disclosure.

FIG. 2 schematically illustrates a monitoring system according to this disclosure.

### DESCRIPTION

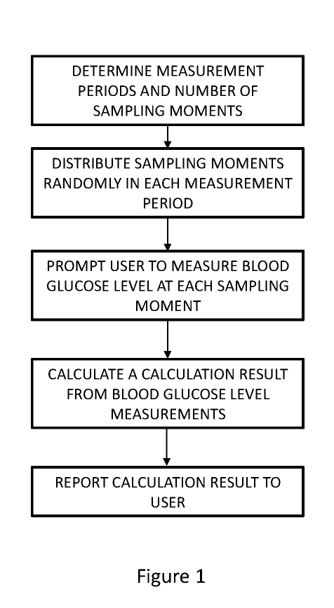

This disclosure relates to a method for sampling blood glucose levels comprising the steps of determining one or more measurement periods, distributing one or more sampling moments randomly within each measurement period and prompting a user to measure the blood glucose level at each sampling moment. The steps of this method are illustrated in FIG. 1.

A measurement period is a time interval characterized by a start time and a stop time. In other words, the word “period” does not refer merely to the length of the time interval between the start time and the stop time, but also to the start and stop times themselves. For example, a measurement period starting at 8 o'clock in the morning and ending at 8 o'clock at night is not the same as a measurement period starting at 9 o'clock in the morning and ending at 9 o'clock at night. The terms “start time” and “stop time” comprise a specific day and the time of day.

Several momentary blood glucose level measurements may be conducted within one measurement period. The user who performs the measurements is not prompted to conduct any momentary blood glucose level measurements outside of a measurement period, but she or he may nevertheless conduct such measurements at any time. The measurement data obtained through such unprompted measurements is stored. This data may or may not be used in the calculations, as described in the two examples below. The decision to include or exclude such additional data in the calculation will depend on data quality and reliability considerations discussed below.

The measurement period or periods can be determined directly by the monitoring system, or through a suggestion provided by the monitoring system and approved by the user, or through free selection by the user. The user's freedom of choice in the determination of measurement periods may sometimes have to be restricted for reasons of quality and reliability. For instance, a very short measurement period will reduce the utility of randomization, especially if the same start and stop times are repeated from one day to the next. The measurement period may then overlap with a regularly recurring event in the patient's daily routine, which means that any averages calculated from the measurements may be influenced by systematic errors.

The length of each measurement period should therefore exceed a certain minimum value, such as 12 hours, but preferably 15 hours. A measurement period may often be shorter than one day because most users do not want to perform momentary blood glucose measurements at inconvenient hours, such as in the middle of the night. Measurement periods therefore typically alternate with quiet periods where the user prefers that no prompts should be given. However, the measurement period can also be longer than one day if the user who will perform the measurement accepts the inconvenience of night-time measurements. There is no upper limit for the length of a measurement period.

In this disclosure the term “total sampling period” refers to the time period from which the data for a calculation has been gathered. The total sampling period usually includes several measurement periods interspersed with quiet periods. If the measurement period is simply one long period without interruptions, the total sampling period may be equivalent to the measurement period.

The number of times that momentary blood glucose levels will be measured in each measurement period can also be determined directly by the monitoring system, or through a suggestion provided by the monitoring system and approved by the user, or through free selection by the user. One measurement time will be called a “sampling moment” in this disclosure. The user who will perform the measurements can select the number of sampling moments relatively freely. Increasing the number of sampling moments within each measurement period will increase the reliability of all calculations.

The number of sampling moments may vary from one measurement period to another. The user may, for example, select the total number of sampling moments which should occur during the total sampling period, and these sampling moments may then be distributed randomly across all measurement periods. However, the number of sampling moments per day should preferably be approximately constant. The number of sampling moments per day should also exceed a certain minimum value. This minimum value may be as low as one, but it should preferably be at least three.

After the measurement periods and the number of sampling moments have been determined, they are transmitted to a control unit in a computer. The control unit then distributes the selected number of sampling moments randomly within each measurement period or across all measurement periods. This distribution can, for example, be performed with the help of a random number generator by retrieving for each sampling moment a separate random number between 0 and 1 from the random number generator. The number 0 can, for example, represent the start time and the number 1 to represent the stop time of a measurement period. Each sampling moment is placed at the time indicated by its random number.

The random number generator is invoked separately for each sampling moment. In other words, sampling moments are not distributed randomly in one measurement period and then copied to other measurement periods.

A sampling moment is a time window characterized by a begin time and an end time. Momentary blood glucose level measurements should be performed in the time window in order for them to be considered valid. In this disclosure, “valid” means simply that the measurement result was collected at a randomly chosen sampling moment. Additional quality checks may also be implemented. The user who performs the measurement is not notified in advance about impending or future sampling moments. The length of the sampling moment window may, for example, be 30 minutes, but preferably 15 minutes. A narrow sampling moment window is important for ensuring that the user does not perform actions after a prompt which would change the momentary blood glucose level before it is measured.

The sampling moment can be implemented as a forward-looking window. In this case, the user who will perform the measurement is prompted at the begin time to perform a measurement before the end time. If a measurement result is received before the end time, it is stored as a valid measurement. Measurement results received after the end time are not considered valid. They may be stored, but they are normally not used in the calculations presented in this disclosure.

Alternatively, the sampling moment can be implemented as a backward- and forward-looking moment. In this case, the user who will perform the measurement is prompted at a predetermined prompt time to perform a measurement. The prompt time is after the begin time but before the end time. If, for some reason, the user has just conducted a blood glucose measurement at a time which is after the begin time but before the prompt time, then the prompt will be cancelled and the measurement result will be stored as a valid measurement.

The total sampling period can be implemented as a sliding time window from which the oldest sampling moments and measurement results are discarded for the next calculation, as new ones are added. It can also be implemented as a continuously expanding time window with a fixed beginning, where new sampling moments and measurement results are added in the present but none are discarded. It can also be implemented simply as a fixed time window with a fixed beginning and end. Other forms of total sampling periods are also possible, and the best implementation will depend on the application.

The greater the total number of sampling moments, the more reliable the resulting calculations will be. The user may not be able or willing to perform a measurement at every prompt, or even within every measurement period. It is preferable that no measurement periods should lack valid measurement data, but certain discrepancies can be accommodated. Even so, a calculation result may not be output if the fraction of measurement periods which lack valid data exceeds a certain threshold value. For instance, no calculation result may be presented if five of the previous ten days lack valid measurement data. Similarly, a calculation result may not be output if the average number of sampling moments within the measurement periods does not exceed a certain threshold value.

This disclosure also relates to an apparatus which is a monitoring system comprising an interface unit, a blood glucose level measurement device and a control unit. Data analysis is performed in the monitoring system and a calculation result is reported to the user through the interface unit.

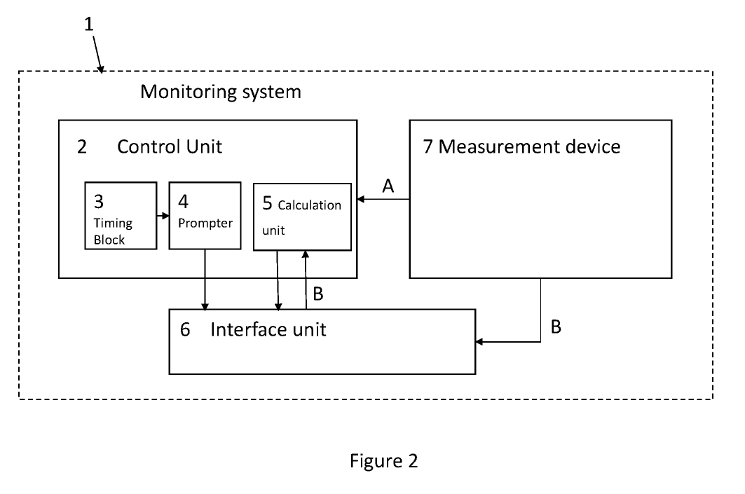

FIG. 2 illustrates this monitoring system 1 schematically. The monitoring system 1 comprises a control unit 2, a blood glucose level measurement device 7 and an interface unit 6. The control unit 2 and the measurement device 7 can either be physically integrated or separated. This is indicated in FIG. 2 where monitoring system 1 has been drawn with a dotted line. If the control unit 2 and the measurement device 7 are integrated, then they may utilize a common interface unit 6. If they are separated, they may have separate interface units. Interface unit 6 in FIG. 2 represents both of these alternatives.

Momentary blood glucose levels can be transferred from the blood glucose level measurement device 7 to the control unit 2 either directly (route A) or via the interface unit 6 (route B). In the latter case the measurement result is output from the measurement device 7 to the interface unit 6, read by the user and then entered into the control unit 2 by the user.

The control unit 2 comprises a timing block 3 and a prompter 4 which are configured to perform the functions described above. The control unit 2 also comprises a calculation unit 5 which is configured to perform the calculations described in the examples below. The calculation unit 5 is also configured to output calculation results to the interface unit 6 for monitoring. This monitoring may include regular checking of estimated values and their historical development by the patient, by another user who assists the patient, or by doctors or other medical personnel.

The timing block 3, prompter 4 and calculation unit 5 may be computer program segments executed either with the same data processors in one hardware unit or with separate data processors in multiple hardware units.

The control unit 2 is adapted to perform the method by giving instructions and prompts to the user through the interface unit 6, optionally by communicating with the measurement device 7, and by receiving data input and performing calculations based on this data.

The blood glucose level measurement device 7 may for example be a blood glucose meter utilizing disposable reagent strips. The measurement device 7 may be suitable for home use, either by a diabetes patient or by another user who assists the diabetes patient in the measurement. The measurement device 7 comprises blood glucose measurement means for determining the glucose level in a blood sample. In addition to measurement means, the measurement device 7 may comprise a processor and output and input units for facilitating user interaction. When taking a blood glucose measurement, the user may first insert one end of a plastic, disposable reagent strip into an electronic measuring device. The user may then apply a small blood sample to the opposite end of the reagent strip. The glucose contained in the blood sample electrochemically reacts with the reagent in the strip, producing an electrical current which is proportional to the glucose concentration in that blood sample. The measuring device 7, within a few seconds, measures and converts to digital format the current signal produced by the reaction in the reagent strip. The measuring device 7 then analyzes this digital current signal with a suitable algorithm and may display the momentary blood glucose level to the user or transfer it to the computer device.

The measurement device 7 may comprise communication means for transferring measurement results automatically to the control unit 2, for example a wireless data link such as, for example, Bluetooth, Wifi, GSM/3G/4G, or a wired data link. As already mentioned, the methods of the present disclosure can also be implemented with a measurement device which does not comprise means for communicating with the control unit. In this case the user must read the measurement result from the measurement device and personally transfer the result to the control unit through the interface unit.

The control unit 2 may be a part of a computer device, and the computer device may be integrated with the measurement device 7 or physically separate from the measurement device 7. The computer device may be a mobile phone, tablet computer, personal computer or the like, adapted to perform the methods of this disclosure.

The control unit 2 may comprise one or more data processors. The control unit may be connected to a memory unit where computer-readable data or programs can be stored. The memory unit may comprise one or more units of volatile or non-volatile memory, for example EEPROM, ROM, PROM, RAM, DRAM, SRAM, firmware, programmable logic, etc.

The interface unit 6 may comprise displays, keyboards, touchscreens, microphones, loudspeakers, or other devices which facilitate user interaction. The control unit 2 and the blood glucose level measurement device 7 are electrically interconnected with the interface unit 6 to provide means for performing the methods described in this disclosure. The interface unit 6 can for example be used to communicate questions, prompts, information or calculation results to the user. It can therefore be used to determine the user's preferences with regard to measurement periods and the number of sampling moments per day, or to acquire measurement results entered by the user. The monitoring system 1 may also comprise communication means for automatically transferring data between the control unit 2 and the measurement device 7 without user intervention, for example a wireless data link such as Bluetooth, Wifi, GSM/3G/4G or a wired data link.

The methods described in the present disclosure may be implemented in, for example, hardware, software, firmware, special purpose circuits or logic, a computing device or some combination thereof. Software routines, which may also be called program products, are articles of manufacture and can be stored in any apparatus-readable data storage medium, and they include program instructions to perform particular predefined tasks. Accordingly, embodiments of this invention also provide a computer program product, readable by a computer and encoding instructions for performing the methods described in this disclosure.

As mentioned, the control unit 2 in the monitoring system includes a timing block 3, a prompter 4 and a calculation unit 5. The timing block 3 is configured to determine one or more measurement periods and distribute one or more sampling moments randomly within each measurement period. The prompter 4 is configured to generate, at each randomly distributed sampling moment, a prompt message through the interface unit 6 which prompts a user to measure a momentary blood glucose level with the blood glucose level measurement device. The calculation unit 5 is configured to use the momentary blood glucose level data to estimate the value of the concentration of glycated hemoglobin (HbA1c) in blood, the long-term blood glucose level, or the blood glucose variance. The calculation unit 5 is also configured to output the estimated value for monitoring through the interface unit 6.

The sampling process begins when the user starts the computer program. In order to determine the measurement periods, the timing block 3 may, for example, suggest to the user a measurement period which covers one day and ask the user to exclude those hours of the day which are inconvenient for performing blood glucose measurements. The measurement periods can be determined in many other ways as well, with a certain degree of free choice by the user, as indicated above. The user may then, for example, be asked to determine a suitable number of sampling moments in each measurement period. The timing block 3 may alternatively determine both the measurement periods and the number of sampling moments autonomously when the user starts the program, with no user input.

Once the number of sampling moments has been determined, the timing block 3 in the control unit 2 distributes the sampling moments randomly within each measurement period in the manner described above. The user may be informed through the interface unit 6 that the distribution has been performed, but details of the distribution are not presented to the user. In particular, the user is not informed about the exact timing of the next sampling moment before being prompted to perform the measurement.

When a sampling moment arrives, the control unit 2 prompts the user to perform a blood glucose measurement through the prompter 4, by presenting a visible and/or audible prompt message to the user through the interface unit 6. The prompt message may be a request to perform a blood glucose measurement. This prompt can be given either at the begin time of the sampling moment, or at a later prompt time, as described above. The end time of the sampling moment may be indicated in the prompt message, but it can also remain unknown to the user. Additional sampling instructions may also be conveyed to the user in the prompt message. These instruction may, for example, request that the patient should perform the measurement before taking any actions which may alter the blood glucose level.

When receiving a prompt, the user performs a measurement with the blood glucose measurement device 7 (presuming that a measurement can be performed). The measurement result may then be transferred to the control unit 2 directly without user intervention. Alternatively, the user may enter the result to the control unit 2 through the interface unit 6.

The measurements performed at randomly distributed sampling moments may not be the only blood glucose measurements which the user performs. The prompts given at sampling moments may not be the only prompts which the computer program gives to the user. The user may, for example, be allowed to set additional prompts at freely chosen moments.

The calculations presented in the examples below should preferably be performed only with data from randomly distributed sampling moments. However, this data can in some embodiments be combined with measurement data obtained from other measurements of momentary blood glucose level which the user has performed according to his or her own schedule. This increase in the number of data points can potentially improve the reliability of the calculation by increasing the number of data points, but precautions must be taken to ensure that the systematic errors discussed in the background section do not influence the calculation results. The additional data may, for example, be limited only to measurements performed in the morning before breakfast. These so-called fasting glucose levels generally correlate more strongly with long-term blood glucose levels than glucose levels measured at other hours of the day.

It has already been indicated above that the reliability of all calculation results is greater if the measurement periods are long. The greatest reliability is obtained if the measurement period is one unitary period extending over the course of several months. This requires that the user accepts sampling moments which occur in the middle of the night. Most users may prefer to divide the total sampling period into a set of measurement periods separated by quiet periods where no sampling moments occur. The night may a preferred quiet period for most users.

It was also indicated above that the calculation results may be considered reliable only if certain threshold values relating to the fraction of measurement periods with valid data, and to the number of sampling moments within each measurement period, are exceeded. Other factors which influence the reliability of the estimates include the number of unprompted blood glucose measurements included in the calculation (if any) and the length of the sampling moment time window. A shorter time window provides a more reliable estimate because it reduces the risk that the periodically recurring daily habits of the user or patient influence the measurement result.

All of these considerations about the reliability of the calculation result can be presented to the user, either as a numerical value or as a simplified scale of low, medium, high. An indicator of the expected reliability can be presented to the user every time a new estimate is presented. An indicator of the expected reliability can also be presented to the user as he or she determines the measurement time periods and the number of sampling moments for a future sampling sequence, so that the reliability consequences of the selections are immediately visible. This reliability indicator may in both cases be expressed as a confidence interval. The user may, for example, be informed after a calculation that there is a 95% probability that the patient's HbA1c-value lies within a certain interval.

Once a sufficient number of valid measurements has accumulated over a sufficiently long measurement period, the program performs calculations and presents the calculation result to the user. These calculation results may be continuously updated as new measurement data are obtained. Past calculation results and time trend graphs may be displayed to the user. Calculation results may also be automatically transmitted through computer networks to other concerned parties, such as the patient's doctor, other medical personnel or relatives.

### Example 1

A first embodiment of the method and apparatus according to this disclosure is a calculation where the momentary blood glucose data obtained with the sampling method described above is used in a calculation which produces as a calculation result an estimate of the long-term blood glucose level and/or an estimate of the HbA1c concentration.

When a satisfactory set of valid measurement data has been obtained, an estimate of the long-term blood glucose level can be calculated by calculating the average of all valid measurement results obtained until then. The long-term blood glucose level is usually calculated as a moving average, which means that new data is added to the average as they are measured, and old data is correspondingly discarded at the other end. In other words, the total sampling period may be implemented as a sliding time window in these calculations. Its length may be selected by the user. Other forms of calculating and presenting the long-term blood glucose level will be obvious to a person skilled in the art.

The data sets from which HbA1c concentration estimates are calculated should preferably cover a total sampling period of 2-3 months. This corresponds to the lifetime of a red blood cell, so a longer total sampling period will not improve the accuracy of the HbA1c estimate. Again, the total sampling period may be implemented as a sliding time window as updated values are calculated. Historical data on how the HbA1c concentration estimate has changed over time can of course be stored over an indefinitely long period. Shorter total sampling periods than 2-3 months may also be used. A first estimate of both the long-term blood glucose level and the HbA1c concentration can usually be presented to the user a few days after sampling has begun. The accuracy of the estimate will of course improve when the total sampling period becomes longer (up to 2-3 months) and additional data is added.

An estimate of the HbA1c concentration can be calculated by weighting each valid measurement result with a time-dependent coefficient, calculating the weighted average and transforming the weighted average into an HbA1c concentration with a transformation table. The time-dependent coefficient may increase linearly as a function of the proximity of the sampling moment to the day of the calculation.

For example, the time-dependent coefficient may be zero for a measurement result obtained three months ago and increase linearly from there up to a value of one for a measurement obtained on the day of the calculation. This time-dependence of the weighting coefficients in HbA1c calculations reflects the fact that recent blood glucose levels influence the momentary HbA1c concentration more strongly than the blood glucose levels which are distant in time.

Alternatively, the weighted average glucose level wAG at time tN may be calculated with the formula:

(wAG)N=a·(wAG)N−1+(1−α)·MGN,

Where (wAG)N−1 is the previous weighted average glucose level calculated at time tN−1, and MGN is the momentary glucose level measured at time tN. The weight of all the previous measurements is a and the weight of the newest measurement is 1−α. The relative weight of the newest measurement can thereby be adjusted using the weight a between 0 and 1. The weighting can, for example, depend on the frequency of valid measurements obtained in the past few months. The base level for a must be determined experimentally, and then a values for higher and lower sampling frequencies may be calculated. Another alternative for adjusting the weight of different measurements is to apply a Kalman-filter.

The weighted average glucose level can then be converted into a HbA1c concentration estimate using known formulas, such as:

(CHbA1c)N=((wAG)N+46.7)/28.7,

where (CHbA1c)N is the estimated HbA1c concentration at time tN expressed in %, and the weighted average glucose level is expressed in mg/dl.

Actual laboratory measurements of the HbA1c concentration performed during the total sampling period may be incorporated into the calculation of subsequent estimates of the HbA1c concentration. In other words, they may be combined with momentary blood glucose level measurements obtained at randomly distributed sampling moments to produce a calculation result. This can, for example, be done by letting the HbA1c concentration measured in the laboratory measurement correspond to (CHbA1c)N−1 in the above formulas. As mentioned above, momentary blood glucose level measurements obtained outside of randomly distributed sampling moments may with some restrictions also be included in the calculation.

Other methods known from the prior art for estimating the long-term blood glucose level and/or the HbA1c concentration from momentary blood glucose level data can also be used. HbA1c-data and blood glucose level data obtained from a broader population may be used to tailor the calculation formulas for each user. For example, if a user consistently excludes nighttime hours from the measurement periods, the uncertainties resulting from this recurring data gap may be reduced by incorporating in the calculation formulas for the HbA1c estimate the typical nighttime blood glucose level behavior of the patient's population group.

Actual laboratory measurements of the HbA1c concentration can also be used to adjust the formula by which the HbA1c is estimated from the sampled momentary blood glucose levels. A discrepancy between the laboratory measurement and the estimated HbA1c value may, for example, be interpreted as an indication that the patient's blood glucose level is not at the expected level during the hours of the day which fall outside of the measurement periods (usually nighttime hours). The formula may be adjusted accordingly. Occasional nighttime measurements may also be suggested to the user to improve reliability.

The benefits obtained with the method and apparatus of the present disclosure do not depend on any particular calculation formula for estimating the long-term blood glucose level and/or the HbA1c concentration. Instead, the benefits are achieved in the sampling stage, in the random distribution of sampling moments which precedes the calculation. The benefit of randomization is that systematic errors resulting from the patient's daily schedule and routines are avoided. Calculation results from data obtained with this sampling method is therefore more reliable than estimates calculated from data obtained with other sampling methods.

### Example 2

A second embodiment of the method and apparatus according to this disclosure is a calculation where the momentary blood glucose data obtained with the sampling method described above is used in a calculation which produces as a calculation result an estimate of blood glucose variance. The data sets from which these estimates are calculated should cover a total sampling period of at least 1 month, preferably at least 3 months.

A basic blood glucose variance calculation simply involves calculating the variance of valid measurement results when a set of measurement data has been obtained. This calculation result is an estimate of blood glucose variance. The estimate can be calculated as a moving average, or as an average over the total sampling period. The total sampling period can be of any length and it can be selected by the user.

Another aspect of blood glucose variance is its short-term variance. The associated calculation result and estimate of blood glucose variance may be an ambulatory glucose profile calculated from the sampled blood glucose levels. As explained in the introduction, an ambulatory glucose profile commonly shows both the variance and the median of momentary blood glucose levels as a function of the time of day when the measurements were obtained. Assuming that the time resolution in the profile is one hour, so that each day is divided into hourly profile intervals, a satisfactory set of valid measurement data may require, for example, a minimum of 5, but preferably at least 10 measurements from each hour of the day. The measurements that fall into one hourly interval may be obtained on different days, but must be within the same hour. The time resolution in the profile may be increased by making the profile intervals shorter, but a longer total sampling period will then be required to collect a sufficient number of measurements in each profile interval.

If, for example, the average number of sampling moments is three per day with no quiet periods, a total sampling period of 3 months would on the average yield approximately 270/24≈11 measurements in each hourly profile interval, which is sufficient for estimating the ambulatory glucose profile with reasonably accuracy. A greater number of sampling moments per day will improve the accuracy of the profile. Alternatively, a greater number of sampling moments per day may allow a shorter total sampling period, so that the ambulatory glucose profile estimate can be updated more often. Alternatively, a greater number of sampling moments may allow a finer time resolution.

The advantage of random sampling over any form of regular sampling schedule, including sampling schedules determined by the user, is that the blood glucose level may undergo variation which may go undetected with a regular sampling schedule. A regular sampling schedule may, of course, sometimes prompt a patient to fill gaps in the sampling schedule with measurements conducted at a specific hour. But if such prompts are always given exactly at the turning of the hour, i.e. at 10.00, 11.00, 12.00 etc., then the hourly variation occurring between the prompt times will go undetected. If the patient routinely eats breakfast and lunch at regular hours, this variation can be significant. As already emphasized earlier, randomly distributed sampling moments allow even hourly variance to be detected more reliably because randomization removes the possibility of a systematic, regular relationship between the sampling schedule and the patient's daily routines.

The data obtained with the sampling method and monitoring system presented in this disclosure can also be used for estimating other calculation results related to blood glucose variance. As explained in conjunction with the first example, the benefits described above are not dependent on any particular calculation formula.

1-12. (canceled)

13. A method for sampling blood glucose levels, the method comprising:
determining one or more measurement periods;
distributing one or more sampling moments randomly within each measurement period; and
prompting a user to measure a momentary blood glucose level at each sampling moment.

14. The method of claim 13, wherein the user is not informed about an exact timing of a next sampling moment before being prompted to perform the measurement.

15. The method of claim 13, wherein each sampling moment is a time window with a begin time and an end time.

16. The method of claim 15, wherein the user is prompted to perform the measurement at a prompt time which equals the begin time.

17. The method of claim 15, wherein the user is prompted to perform the measurement at a prompt time which is after the begin time but before the end time.

18. A monitoring system, comprising:
an interface unit;
a blood glucose level measurement device; and
a control unit that includes a timing block, a prompter and a calculation unit, wherein:
the timing block is configured to determine one or more measurement periods and distribute one or more sampling moments randomly within each measurement period,
the prompter is configured to generate, at each randomly distributed sampling moment, a prompt message through the interface unit which prompts a user to measure a momentary blood glucose level with the blood glucose level measurement device,
the calculation unit is configured to use the momentary blood glucose level data to estimate the value of the concentration of glycated hemoglobin (HbA1c) in blood, the long-term blood glucose level, or the blood glucose variance, and
the calculation unit is configured to output the estimated value for monitoring through the interface unit.

19. The monitoring system of claim 18, wherein the timing block is configured to use a random number generator to distribute the sampling moments within each measurement period.

20. A computer program product readable by computer, the computer program product being configured to encode instructions to perform a method for sampling blood glucose levels by:
determining one or more measurement periods;
distributing one or more sampling moments randomly within each measurement period; and
prompting a user to measure a momentary blood glucose level at each sampling moment.
